(19) United States
(12) Patent Application Publication
(10) Pub. No.: US 2025/0239338 A1
(43) Pub. Date: Jul. 24, 2025

(54) Method for Operating an Imaging Apparatus, an Imaging Apparatus, a Computer Program, and an Electronically Readable Data Carrier

(71) Applicant: Siemens Healthineers AG, Forchheim (DE)

(72) Inventors: Philipp Höcht, Lauf (DE); Mario Bacher, Diepersdorf (DE)

(73) Assignee: Siemens Healthineers AG, Forchheim (DE)

(21) Appl. No.: 19/033,827

(22) Filed: Jan. 22, 2025

(30) Foreign Application Priority Data
Jan. 23, 2024 (DE) 10 2024 200 587.8

(51) Int. Cl. G16H 10/60; G06V 40/20

(52) U.S. Cl. CPC G16H 10/60; G06V 40/20

(57) ABSTRACT

The disclosure relates to techniques for operating an imaging apparatus, which includes receiving sensor data via a control facility of the imaging apparatus. The sensor data describes sensor values relating to a patient to be examined. The technique further includes determining a state of the patient to be examined by the control facility based on the sensor data, and checking the state of the patient to be examined for fulfillment of a predetermined recording condition for carrying out a recording method by a recording facility of the imaging apparatus. Upon fulfillment of the predetermined recording condition, a recording facility is triggered by the control facility to carry out the recording method, and a recording is performed by the recording facility. The recording method comprises recording at least one recording of a recording area of the patient to be examined.

### CROSS-REFERENCE TO RELATED APPLICATIONS

The present application claims priority to and the benefit of Germany patent application no. DE 10 2024 200 587.8, filed on Jan. 23, 2024, the contents of which are incorporated herein by reference in their entirety.

### TECHNICAL FIELD

The disclosure relates to a method for operating an imaging apparatus, an imaging apparatus, a computer program, and an electronically readable data carrier.

### BACKGROUND

Imaging apparatuses, such as magnetic resonance imaging apparatuses or computed tomography apparatuses, are used to record two and/or three-dimensional recording data of a recording area of a patient to be examined. A recording method to be carried out by the imaging apparatus for recording the recording data according to conventional techniques is typically initiated manually by an operator of the imaging apparatus as soon as the workflow permits. The quality of the recording data depends on the extent to which the patient lies still at least in the recording area. For this reason, it is important that the patient avoids movements during recording as much as possible.

These movements may include respiratory movements of the patient, such as movements related to the breathing of the patient. To minimize the effects of the respiratory movements of the patient on the recording data, it is customary for the patient to be given instructions through the imaging apparatus so that they can adjust their breathing accordingly. For instance, it is desirable for the patient to exhale before the recording so that during the recording they are in a respiratory phase in which they have exhaled and not making any inhalation or exhalation movements.

At present, however, instructions are given without taking into account the respiratory characteristics of the patient. Such respiratory characteristics can, for example, comprise the respiratory rate and/or the current phase of respiration of the patient. This can lead to the imaging apparatus prompting the patient to inhale even though the patient has just started exhaling. This forces the patient to deviate from their natural breathing rhythm. In this case, the patient would exhale faster and inhale again immediately.

This can be perceived as unpleasant by the patient, and may also have a negative impact on the quality of the recording data. If the patient is forced to deviate from their customary breathing rhythm, the time during which they can hold their breath is often reduced and/or they move as a result of the restlessness caused by holding their breath for too long. Due to the resulting movements, the desired optimization of the recording quality, which is the aim of the instructions, cannot be achieved.

The only way to be able to respond to the respiratory characteristics of the patient is for the operator of the imaging apparatus to monitor the patient and give the instructions to the patient and initiate the recording process manually. However, this means additional work for the operator.

### SUMMARY

It is an object of the disclosure to adapt a recording method of an imaging apparatus to the breathing of a patient to be examined.

This object is achieved by the embodiments as discussed herein, including the claims.

A first aspect of the disclosure relates to a method for operating an imaging apparatus. The imaging apparatus may be, for example, an magnetic resonance imaging (MRI) apparatus or a computed tomography (CT) apparatus. In the method, it is provided that sensor data is received by a control facility of the imaging apparatus. The sensor data comprises sensor values that describe a patient to be examined. The sensor values may, for example, describe recorded movements of the patient. The sensor values may comprise, for example, a value that describes a movement of a chest of the patient.

One step comprises the determination of a state of the patient to be examined by the control facility based on the sensor data. In other words, the state of the patient to be examined may be determined by the control facility. The state of the patient may, for example, relate to a respiratory characteristic of the patient. For example, it may be provided that the state describes a breathing rhythm of the patient and/or a current phase of respiration of the patient. For example, it may be provided that the sensor data describes the movement of the chest of the patient. The control facility may be configured to track the movement of the chest over a period of time determine the breathing rhythm of the patient and/or the current phase of respiration of the patient.

One step comprises checking the state of the patient to be examined for fulfillment of a predetermined recording condition for carrying out a recording method by a recording facility of the imaging apparatus. In other words, it is provided that, after the determination of the state of the patient, the control facility checks whether the determined state of the patient fulfills the predetermined recording condition. The recording condition may specify a state of the patient, which is required to initiate the recording method by the recording facility. For example, it may be provided that the recording condition provides that the patient is in a certain phase of their breathing rhythm. For example, it may be necessary for the recording condition to provide that the patient has completed a phase of exhalation.

It is provided that when the predetermined recording condition is met, the recording facility is controlled by the control facility for carrying out the recording method. In other words, the method comprises triggering of the recording facility by the control facility to carry out the recording method when the predetermined recording condition is met.

The method comprises carrying out the recording method by the recording facility, the recording method comprising recording at least one recording of a recording area of the patient to be examined. In other words, it is provided that the recording facility carries out the recording method when it is activated by the control facility. In the recording method, the recording facility records at least one recording of the recording area of the patient to be examined. The recording method can thus comprise recording one or more recordings of the recording area. For example, the respective recordings can be recorded from different angles.

The disclosure has the advantage that it can be ensured that the recording method is only initiated when the recording condition is met. This makes it possible to ensure that the recording method is only carried out when the patient is no longer moving, for example.

The disclosure also comprises developments which result in further advantages.

One development of the disclosure provides that the method performs the checking of the state of the patient to be examined for the fulfillment of a predetermined instruction condition for the performance of an instruction method by an output facility of the imaging apparatus. In other words, the control facility checks whether the state of the patient fulfills the predetermined instruction condition. The instruction condition can specify a state of the patient that is required for the instruction method to be initiated by the control facility. When the predetermined instruction condition is fulfilled, the output facility is triggered by the control facility to issue instructions to the patient. The instruction method comprises the triggering of the output facility by the control facility to issue instructions to the patient. The instructions are intended to guide the patient so that the recording condition is fulfilled. In other words, the output facility is triggered when the predetermined condition is fulfilled by the control facility. For example, it may be provided that the instruction condition requires that the respiratory characteristic of the patient has a respiratory rate within a predetermined range of values and/or that a predetermined phase of respiration of the patient is present. The instructions may, for example, prompt the patient to inhale, exhale, or to hold their breath. The instructions may, for example, depend on a current phase of respiration of the patient and may be intended to influence the respiratory rate of the patient and/or a phase of respiration so that it fulfills the recording condition.

One development of the disclosure provides that the method comprises logging the state of the patient over a specified period. In other words, it is provided that the state of the patient is recorded several times during the specified period. For example, it may be provided that the respiratory state of the patient is recorded several times during the time period. The specified time period may be prior to the actual examination, during which the state of the patient is used to determine whether the instruction condition and/or the recording condition(s) are fulfilled.

The method comprises a determination of a patient characterization of the patient based on the state of the patient logged during the specified period. In other words, the patient is characterized based on the conditions of the patient recorded during the specified period. For example, it may be provided that the respiratory conditions recorded during the period are evaluated to determine a general respiratory characteristic of the patient. The general respiratory characteristic may, for example, describe a respiratory rate and/or the duration of certain phases of respiration.

One development of the disclosure provides that the method comprises the determination of the recording condition depending on the patient characterization of the patient. In other words, the recording condition is based on the patient characterization. For example, it may be provided that a phase of respiration is selected for carrying out the recording depending on the respiratory characteristics. For example, it may be provided that, depending on the length of a phase of inhalation and the length of a phase of exhalation, it is determined according to the patient characterization which of the phases is selected for recording. The advantage of the development is that the recording condition can be adjusted depending on the respective patient.

One development of the disclosure provides that the method includes a determination of the instruction condition depending on the patient characterization of the patient. In other words, the instruction condition for issuing the instructions is determined on the basis of the patient characterization. For example, it may be provided that a change in the respiratory rate of the patient is determined on the basis of the patient characterization. The change in the respiratory rate may be used, for example, to estimate how long it will take until the respiratory rate of the patient approaches a resting respiratory rate. Based on this, the instruction condition can be selected. Thus, the issuing of instructions may, for example, only take place when the breathing of the patient has approached a target resting respiratory rate, and it can therefore be assumed that the respiratory rate will remain constant. If the instructions are issued too early, however, they could not be implemented due to the changing respiratory rate of the patient.

One development of the disclosure provides that the issuing of the instructions to the patient for guiding the patient is dependent on the patient characterization of the patient. In other words, the issuing of the instructions to the patient is adjusted based on the patient characterization. That is, the method not only ensures that the recording is carried out when the recording condition is met, but also ensures that the instructions are adapted to the respective patient. For example, it may be provided that verbally-issued instructions comprising the outputs “Breathe out—Breathe in—Breathe out—Hold your breath” are spread out over time or condensed depending on the breathing rhythm of the patient so that the frequency with which the instructions are issued is adapted to the respiratory rate of the patient. The instructions can be stored in a pre-recorded audio file, which may be adapted using corresponding audio stretch mechanisms according to the respiratory rate of the patient. Alternatively, it is possible to issue the instructions via speech synthesis in the language of the patient. By adapting the issuing of the instructions, improved guidance of the patient can be made possible.

One development of the disclosure provides that the instructions comprise audio signals. In other words, it is provided that the control facility controls the output facility to output sounds or voice signals. The development has the advantage that the patient can be guided acoustically.

One development of the disclosure provides that the instructions comprise visual representations. In other words, the control facility is configured to control the output facility to output visual representations. It may be provided that the visual representations depict a pattern of breathing which is to be emulated by the patient. The advantage of the development is that the patient can be guided visually.

One development of the disclosure provides that the state comprises a movement characteristic of the patient. In other words, the movement characteristic of the patient is determined by the control facility from the sensor data. The movement characteristics of the patient may, for example, relate to a movement of certain anatomical areas of the patient. The movement characteristics may, for example, relate to movements of the eyelids, limbs, or certain muscles of the patient. The movement characteristics may, for example, relate to swallowing movements. As an example, the movement characteristics may relate to those movements of the patient which may be consciously influenced by the patient. This has the advantage that the movement of the patient can be influenced so that they can adapt their movement characteristics to the examination.

One development of the disclosure provides that the state comprises a respiratory characteristic of the patient. In other words, it is intended to monitor the breathing of the patient and/or to characterize the breathing of the patient. The monitoring of respiration is particularly advantageous because a deviation of a respiratory phase of the patient from a required phase during recording may lead to errors in the representation.

One development of the disclosure provides that the method comprises the recording of the sensor values relating to the patient to be examined by a sensor facility of the imaging apparatus. In other words, it is provided that the imaging apparatus has a sensor facility, which is configured to record the sensor values of the patient. The sensor facility may, for example, comprise a camera that is configured to record images of the patient. A further step comprises the transmission of the sensor data comprising the sensor values to the control facility of the imaging apparatus by the sensor facility. The advantage of the development of the disclosure is that the imaging apparatus itself records the sensor data. It is therefore not necessary to use an external apparatus to provide the sensor data.

One development of the disclosure provides that the sensor facility comprises a pilot tone navigation facility (also referred to herein as a pilot tone navigator). In other words, it is provided that the sensor data is recorded by means of the pilot tone navigation facility of the sensor facility. A pilot tone navigation facility may, for example, be identical or similar to that disclosed in the unexamined patent application DE 10 2015 224 158 A1 (also see U.S. Pat. No. 10,393,845).

For use cases or use situations that may arise with the method, and which are not explicitly described here, it may be provided that in accordance with the method an error message and/or a request to enter user feedback is output and/or a default setting and/or a predetermined initial state is set.

A second aspect of the disclosure relates to an imaging apparatus. The imaging apparatus may be, for example, a CT apparatus or an MRI apparatus. The imaging apparatus may be designed to carry out any of the methods as described in accordance with the first aspect of the disclosure. Thus, the entire method in accordance with the first aspect of the disclosure, or any individual steps thereof, may be carried out by the imaging apparatus in accordance with the second aspect of the disclosure.

The imaging apparatus may have a control facility that is configured to receive sensor data. Again, the sensor data may comprise sensor values relating to a patient to be examined. The control facility can, for example, comprise an interface at which the sensor data can be provided to the control facility.

The control facility is configured to determine a state of the patient to be examined based on the sensor data. The control facility may, for example, be configured to determine the state of the patient to be examined from current sensor values of the sensor data and/or to determine the state of the patient to be examined based on sensor values of the patient recorded over a period of time.

The control facility is configured to check the state of the patient to be examined for fulfillment of a predetermined recording condition. The predetermined recording condition may include specifications for carrying out a recording method by a recording facility of the imaging apparatus. The recording condition may, for example, specify a required respiratory rate or a required phase of respiration of the patient.

The control facility is configured to control the recording facility of the imaging apparatus upon fulfillment of the predetermined recording condition to carry out the recording method using the recording facility. The recording facility may, for example, comprise one or more X-ray sources and one or more detectors.

The recording facility may be configured to carry out the recording method and/or to record at least one recording of a recording area of the patient to be examined in the recording method.

As an example, the control facility may include one or more computers, one or more microcontrollers, and/or one or more integrated circuits, for example one or more application-specific integrated circuits (ASICs), one or more field-programmable gate arrays (FPGAs), and/or one or more systems on a chip (SoCs). The computing unit may also include one or more processors, for example one or more microprocessors, one or more central processing units (CPUs), one or more graphics processing units (GPUs), and/or one or more signal processors, e.g. one or more digital signal processors (DSPs). The computing unit may also include a physical or a virtual network of computers, or other of the aforementioned units.

A third aspect of the disclosure relates to a computer program, which may be loaded directly into a memory of a control facility and having program means for carrying out any or all of the steps of the aforementioned method when the program is executed in the control facility. Thus, the method described herein may also be in the form of a computer program product, which may implement the method in accordance with the first aspect of the disclosure, or any individual steps thereof, on a control facility when executed on the control facility.

A fourth aspect of the disclosure relates to an electronically readable data carrier with electronically readable control information stored thereon, which comprises at least one computer program described and configured to carry out the method in accordance with the first aspect of the disclosure, or any individual steps thereof when the data carrier is used in a control facility.

The claims, figures, and the description of the figures contain further features of the disclosure. The features and feature combinations mentioned above in the description, and the features and feature combinations mentioned below in the description of the figures and/or shown in the figures can be included by the disclosure not just in the particular combination stated but also in other combinations. In particular, the disclosure can include embodiments and feature combinations that do not have all the features of a claim in the original wording. Furthermore, the disclosure can include embodiments and feature combinations that go beyond or differ from the feature combinations presented in the dependency references of the claims.

### BRIEF DESCRIPTION OF THE DRAWINGS

The disclosure is described in greater detail below with reference to specific exemplary embodiments and associated schematic drawings. In the figures, identical or functionally equivalent elements can be denoted by the same reference characters. The description of identical or functionally equivalent elements is not necessarily repeated when referring to different figures. The figures show:

FIG. 1 illustrates a schematic representation of an exemplary imaging apparatus, in accordance with one or more embodiments of the present disclosure; and

FIG. 2 illustrates an example method flow for operating an imaging apparatus, in accordance with one or more embodiments of the present disclosure.

### DETAILED DESCRIPTION OF THE DISCLOSURE

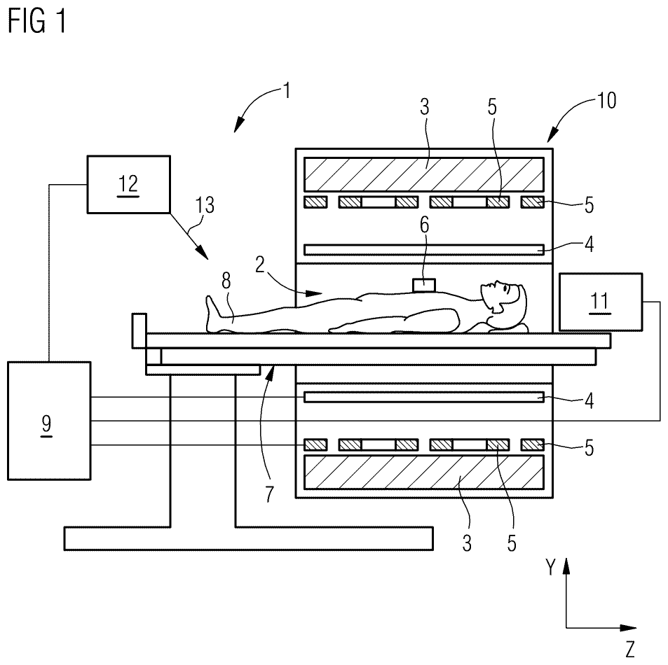

FIG. 1 illustrates a schematic representation of an exemplary imaging apparatus, in accordance with one or more embodiments of the present disclosure.

The imaging apparatus 1 may be implemented as an MRI apparatus, for example. The imaging apparatus 1 comprises a recording facility 10 (also referred to herein as a recorder) with a field magnet 3 (also referred to herein as a main magnet), which generates a static magnetic field for aligning nuclear spins of a patient 8 in an imaging area. The imaging area may be characterized by an extremely homogeneous static magnetic field, the homogeneity relating to the magnetic field strength or its amplitude. The imaging area may be located in a patient tunnel 2, which extends in a longitudinal direction Z through the recording facility 10. The field magnet 3 may be a superconducting magnet, for example, which can generate magnetic fields with a magnetic flux density of up to 3 T or more. For lower field strengths, however, permanent magnets or electromagnets with normally conducting coils may also be used. A patient table 7 may be movable within the patient tunnel 2.

Furthermore, the recording facility 10 comprises a gradient coil arrangement 5 with a plurality of gradient coils, which serve to superimpose gradient fields, i.e. location-dependent magnetic fields, on the static magnetic field in the three spatial directions for spatial differentiation of the scanned image areas in the imaging area. The gradient coils of the gradient coil arrangement 5 may be implemented, for example, as coils made of normally conducting wires which can, for example, generate mutually orthogonal fields or field gradients in the imaging area.

The recording facility 10 comprises a transmitter coil arrangement which can, for example, comprise a body coil 4 as a transmitting antenna, which is implemented to radiate a high-frequency (e.g. RF) signal into the imaging area. The body coil 4 can therefore be understood as an RF transmitter coil arrangement of the recording facility 10 or as part of the RF transmitter coil arrangement. In some embodiments, the body coil 4 may also be used to receive resonant MR signals emitted by the patient 8. In this case, the body coil 4 may also be regarded as part of a signal acquisition apparatus of the recording facility 10. Optionally, the signal acquisition apparatus may comprise a local coil 6, which may be arranged in the immediate vicinity of the patient 8; for example on the patient 8 or in the patient table 7. As an alternative to the body coil 4, the local coil 6 may serve as a receiving coil or a receiving antenna.

The recording facility 10 may be configured to record one or more recordings of the patient 8 in a recording method and to make them available to a control facility 9 (also referred to herein as a controller). The control facility 9 may evaluate the recordings and, for example, perform an image reconstruction.

To be able to ensure a certain recording quality, it may be necessary for the patient 8 to make no movements or as few movements as possible during the recording method. For instance, it may be necessary to ensure that the patient 8 breathes in such a way that no respiratory movements are made by the patient 8 during the recording method. It is advantageous if the breathing of the patient 8 is in a phase in which the patient 8 has exhaled or inhaled and is holding their breath.

For this purpose, the imaging apparatus 1 may include a sensor facility 11 (also referred to herein as a sensor or sensor suite, which includes more than one sensor), and which may be configured to record sensor values of the patient 8 and to transmit sensor data comprising the sensor values to the control facility 9. The sensor facility 11 can, for example, have a camera that monitors the movement of the chest of the patient 8. The control facility 9 may be configured to determine a state of the patient 8 to be examined from the sensor data provided. The state may, for example, comprise a breathing rhythm and/or a phase of the breathing rhythm of the patient 8. The control facility 9 may, for example, determine a respiratory characteristic of the patient 8 from a regular raising and lowering of the chest of the patient 8. The respiratory characteristic may comprise, for example, a respiratory rate of the patient 8. A current phase of the breathing of the patient 8 may be determined by the control facility 9 from a current raising of the chest of the patient 8.

The control facility 9 may be configured to monitor the state of the patient 8 and to trigger the recording facility 10 to record the recording data of the patient 8 when a recording condition is met by the state of the patient 8. For example, it may be provided that the recording condition is met when the patient 8 has completed an exhalation process and respiratory movements are therefore absent for a certain period of time. By carrying out the recording method during this time period, it can be ensured that the recordings are not impaired by the respiratory movements.

However, it may be necessary to guide the patient 8 by means of instructions 13 so that the recording condition is met. For this purpose, the imaging apparatus 1 may have an output facility 12 (also referred to herein as an output interface), which may be configured to issue instructions 13 to the patient 8 when the output facility 12 is controlled by the control facility 9. The instructions 13 may comprise, for example, voice output and/or visual representations. The output facility 12 may be controlled by the control facility 9 depending on the current state of the patient 8. The instruction method can be initiated when an instruction condition is met by the state of the patient 8. For example, it may be provided that a respiratory rate of the patient 8 does not yet meet the recording condition. However, the respiratory rate may lie within a range of values that meets the instruction condition. In this case, the control facility 9 may control the output facility 12 and issue instructions 13, which relate to the breathing pattern of the patient 8. The instructions 13 may be intended to influence the respiratory rate of the patient 8 in such a way that a current respiratory rate of the patient 8 approaches a required (e.g. target) respiratory rate of the patient 8 according to the recording condition.

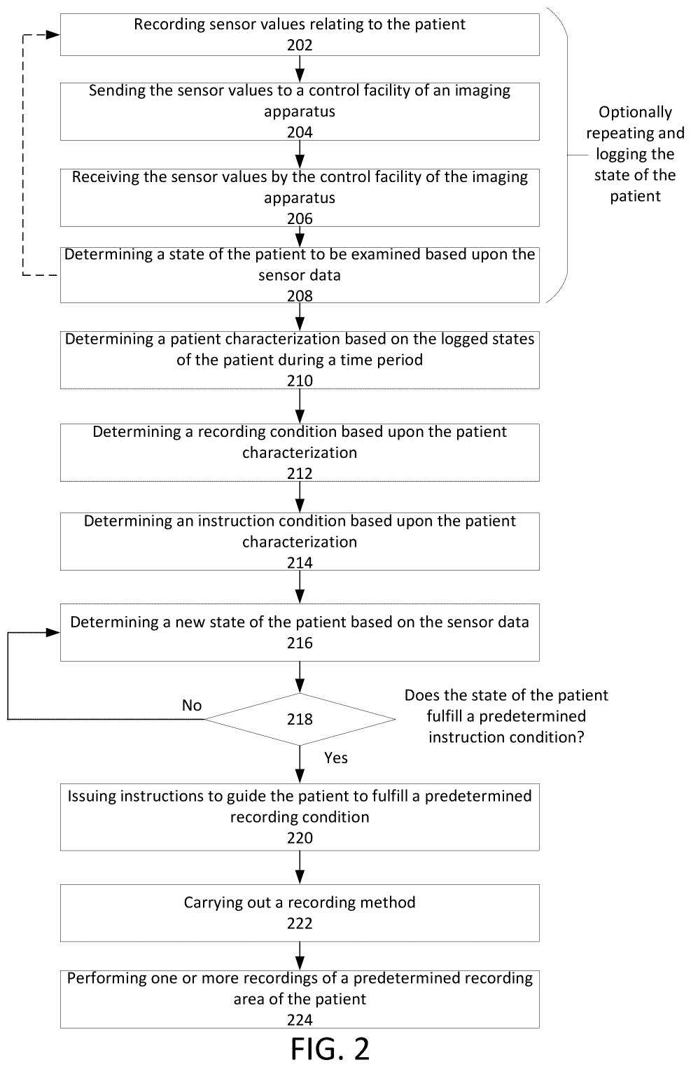

FIG. 2 illustrates an example method flow for operating an imaging apparatus, in accordance with one or more embodiments of the present disclosure. The method as shown in FIG. 2 may be performed, for instance, via the imaging apparatus 1.

A step 202 of the method may comprise recording sensor values relating to the patient 8 to be examined by a sensor facility 11 of the imaging apparatus 1. The sensor facility 11 may comprise, for example, a camera, a belt for breath measurement, a pilot tone-based sensor, combinations of these, etc.

A step 204 of the method may comprise sending the sensor data comprising the sensor values to the control facility 9 of the imaging apparatus 1 by the sensor facility 11. This may include a data transmission via any suitable interface and/or communication techniques.

A step 206 of the method may comprise receiving the sensor data by the control facility 9 of the imaging apparatus 1. This may include receiving a data transmission via any suitable interface and/or communication techniques.

A step 208 of the method may comprise determining a state of the patient 8 to be examined by the control facility 9 based on the sensor data.

Steps 202 to 208 inclusive may be carried out multiple times over a certain period of time. The conditions of the patient 8 (e.g. the state of the patient) determined during the specified period may be logged by the control facility 9.

In a step 210, a patient characterization of the patient 8 can be determined by the control facility 9 based on the logged states of the patient 8 during the certain period of time.

In a step 212, the recording condition can be determined by the control facility 9 depending on the patient characterization of the patient 8.

In a step 214, the control facility 9 can determine the instruction condition depending on the patient characterization of the patient 8.

After the recording condition and the instruction condition have been determined by the control facility 9, a new determination of a state of the patient 8 to be examined can be carried out by the control facility 9 based on the sensor data in a step 216 of the method.

A step 218 of the method can comprise checking the state of the patient 8 to be examined for fulfillment of a predetermined instruction condition for carrying out an instruction method by an output facility 12 of the imaging apparatus 1.

When the predetermined instruction condition is met by the determined state of the patient 8, in a step 220 of the method, the output facility 12 can be controlled by the control facility 9 to issue instructions 13 to the patient 8 for guiding the patient 8 to fulfill the predetermined recording condition. For example, it may be provided that during the instruction method, the state of the patient 8, for example a current phase of the breathing of the patient 8, is monitored and the patient 8 is prompted to inhale or exhale at determined times by means of the instruction 13.

When a predetermined recording condition is met by the state of the patient 8, the recording facility 10 can be controlled by the control facility 9 in a step 222 to carry out a recording method by means of the recording facility 10.

In the recording method, in a step 224, the recording facility 10 can record one or more recordings of a predetermined recording area of the patient 8 to be examined and provide them to the control facility 9.

Although the disclosure has been illustrated and described in more detail on the basis of the exemplary embodiments, the disclosure is nonetheless not limited by the disclosed examples and other variations may be derived herefrom by the person skilled in the art without leaving the scope of protection of the disclosure.

Independent of the grammatical term usage, individuals with male, female or other gender identities are included within the term.

The various components described herein may be referred to as “units.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such units, as applicable and relevant, may alternatively be referred to herein as “circuitry,” “controllers,” “processors,” or “processing circuitry,” or alternatively as noted herein.

What is claimed is:

1. A method for operating an imaging apparatus, comprising:
receiving, via a controller of the imaging apparatus, sensor data describing sensor values relating to a patient to be examined;
determining, via the controller, a state of the patient based on the sensor data;
determining whether the state of the patient satisfies a predetermined recording condition to perform a recording via a recorder of the imaging apparatus;
in response to the predetermined recording condition being satisfied, controlling, via the controller, the recorder to cause the recorder to perform a recording of a recording area of the patient to be examined.

2. The method as claimed in claim 1, further comprising:
determining whether the state of the patient satisfies a predetermined instruction condition; and
in response to the predetermined instruction condition being satisfied, controlling, via the controller, an output interface to provide instructions to the patient to instruct the patient to satisfy the predetermined recording condition.

3. The method as claimed in claim 1, further comprising:
logging the state of the patient over a time period; and
determining a patient characterization of the patient based on the state of the patient logged during the time period.

4. The method as claimed in claim 3, wherein the determining whether the patient satisfies the predetermined recording condition is based upon the patient characterization of the patient.

5. The method as claimed in claim 2, further comprising:
logging the state of the patient over a time period; and
determining a patient characterization of the patient based on the state of the patient logged during the time period, and
wherein the determining whether the state of the patient satisfies the predetermined instruction condition is based upon the patient characterization of the patient.

6. The method as claimed in claim 2, further comprising:
logging the state of the patient over a time period; and
determining a patient characterization of the patient based on the state of the patient logged during the time period,
wherein the instructions provided to the patient are based upon the patient characterization of the patient.

7. The method as claimed in claim 2, wherein the instructions provided to the patient comprise audio signals.

8. The method as claimed in claim 2, wherein the instructions provided to the patient comprise visual representations.

9. The method as claimed in claim 1, wherein the state of the patient comprises a movement characteristic of the patient.

10. The method as claimed in claim 1, wherein the state of the patient comprises a respiratory characteristic of the patient.

11. The method as claimed claim 1, further comprising:
recording the sensor values relating to the patient via a sensor of the imaging apparatus; and
transmitting the sensor data comprising the sensor values to the controller.

12. The method as claimed in claim 11, wherein the sensor comprises a pilot tone navigator.

13. An imaging apparatus, comprising:
a main magnet;
a recorder; and
a controller configured to:
receive sensor data describing sensor values relating to a patient to be examined;
determine a state of the patient based on the sensor data;
determine whether the state of the patient satisfies a predetermined recording condition to perform a recording via a recorder of the imaging apparatus,
wherein in response to the predetermined recording condition being satisfied, the controller is configured to control the recorder to cause the recorder perform a recording of a recording area of the patient to be examined.

14. A non-transitory computer-readable medium having instructions stored thereon that, when executed by a controller of an imaging apparatus, cause the imaging apparatus to:
receive sensor data describing sensor values relating to a patient to be examined;
determine a state of the patient based on the sensor data;
determine whether the state of the patient satisfies a predetermined recording condition to perform a recording via a recorder of the imaging apparatus; and
in response to the predetermined recording condition being satisfied, control the recorder to cause the recorder perform a recording of a recording area of the patient to be examined.
